(19) United States
(12) Patent Application Publication
(10) Pub. No.: US 2005/0182379 A1
(43) Pub. Date: Aug. 18, 2005

(54) Disposable absorbent article having a joined compactly stored disposable storage bag

(76) Inventors: Bradley D. Olsen, Sandy, UT (US); Aaron D. Olsen, Sandy, UT (US)

Correspondence Address: BRADLEY D. OLSEN, 524 EAST 10375 SOUTH, SANDY, UT, 84070, US

(21) Appl. No.: 10/782,597

(22) Filed: Feb. 18, 2004

(51) Int. Cl. A61F 13/15; A61F 13/20

(52) U.S. Cl. 604/385.13

(57) ABSTRACT

A disposable absorbent article(s) with a built-in, self-containing disposal system, which includes a joined and or bonded compactly stored pull-out sealable storage bag within a safety pouch or under an over-layer of material that is protected by a discreet integrated child-resistant safety tab or tape, that is disposed discreetly and directly to the center of the back waist region on an outer surface and/or backsheet of the disposable absorbent article.

### FIELD OF THE INVENTION

The present invention relates to articles which absorb and/or contain bodily exudates, including disposable absorbent articles such as diapers, training pants, pull-ups, swim pants, adult incontinence products, disposable adult protective underwear, undergarments, pantiliners, pantiliner pads and disposable pet diapers and/or undergarments. More particularly, the invention relates to disposable absorbent articles having improved management and/or means for disposal and/or discarding in a more convenient, efficient, economical and environmental compatible way.

### BACKGROUND OF THE INVENTION

The major function of absorbent articles such as diapers and adult incontinence briefs is to prevent body exudates from soiling, wetting, or otherwise contaminating clothing or other articles, such as bedding, that come in contact with the wearer. In recent years, disposable diapers, such as those disclosed in U.S. Pat. No. 3,860,003 issued to Kenneth Barclay Buell on Jan. 14, 1975; and U.S. Pat. No. 5,957,906 entitled “Diaper with improved feces management properties” issued to Roe et al., on Sep. 28, 1999, each individually incorporated herein by reference, have become very popular with the public and have generally replaced durable cloth absorbent articles because of their convenience and reliability. However, the undesirable effects of the absorbent article after use, such as odor and germs that are stored in the diaper remain a problem for the user for proper and adequate disposal or discard within a closed room or any other area where the soiled article has been removed. Generally, as disclosed in U.S. Pat No. 6,267,836 issued to Fenske et al., on Jul. 31, 2001, which is incorporated herein by reference, the diaper tape tabs are utilized, wherein, a parent who changes the used and/or soiled diaper, either rolls and/or folds the diaper up into a ball-like object and then secures the article by re-fastening the diaper tape tabs, prior to discarding it into the trash. This obviously can leave a foul or unpleasant smell throughout the area where the trash is located. Despite the effectiveness of such disposable articles when being worn by a person, the diaper is not designed nor does it have any function that allows it to be stored in an airtight, odor-free and germ-free environment prior to disposal or discard after its use. Thus, several attempts have been made to add features to diapers such as barriers, pockets, spacers, transverse barriers, apertured topsheets and the like to limit the odor and germs and better confine fecal matter in the diaper during its use, however, such attempts have been generally unsuccessful due to their cost and complexity or due to their limited success in reducing the negative effects of the used and/or soiled article. Accordingly, the special characteristics for an environmentally compatible disposal means of viscous fluid bodily wastes need to be addressed by unique acceptance, storage, containment and immobilization of germs and odors in an airtight compartment.

A Description of prior art is the Pampers Diaper, The Huggies Diaper, The Luvs Diaper and Training Pants, Pull-ups, Night-time underwear for children, Adult Protective Underwear, Pantiliners and Pantiliner Pads. The prior art is specific with claims of the function and uses of these products however, the prior art lacks claim or is without sufficient claim, of a convenient, proper and safe means to discard and or disposal of the article after-use in an environmentally compatible manner.

Other prior art in the field to which the invention pertains to a diaper pail or other apparatus of the like used for discarding a soiled absorbent article, such as a diaper, and is disclosed in U.S. Pat. No. 6,612,009 entitled “Waste disposal device including a bag cartridge of flexible tubing” issued to Stravitz on Sep. 2, 2003; U.S. Pat. No. 5,799,909 entitled “Containment system for receiving and disposing of disposable sanitary products” issued to Ziegler on Sep. 1, 1998; U.S. Pat. No. 5,765,339 entitled “Diaper Pail” issued to Garland on Jun. 16, 1998. Each of these incorporated herein by reference. The prior art also states a means or manner of a more sanitary and substantially odorless disposal of articles like a soiled diaper as in other prior art of a diaper pail or diaper disposal system, wherein, products are manufactured and/or sold under Trade Mark Name(s), such as, “The Diaper Genie™” sold by Playtex, “The Diaper Champ™” sold by Baby Trend, and “The Neat Diaper Disposal System™” sold by Safety 1^{st}, are popular with the public, and are disclosed in U.S. Pat. No. 4,869,049 issued to Richards et al, on Sep. 26, 1989; U.S. Pat. No. 5,590,512 issued to Richards et al, on Jan. 7, 1997; U.S. Pat. No. 5,813,200 issued to Jacoby et al, on Sep. 29, 1998; U.S. Pat. No. 6,128,890 issued to Firth on Oct. 10, 2000; and U.S. Pat. No. 6,170,240 issued to Jacoby et al., on Jan. 9, 2001 and respectively, all are incorporated herein by reference. The Diaper Genie™ claims a unique twisting action individually seals diapers in an odor-free, germ-free plastic bag chain within a tower unit which the base opens for easy disposal, easy one-hand operation with child-resistant lock, and holds up to 30 diapers and requires bag refill units. Other prior art, The Diaper Champ claims it holds disposable or cloth diapers, odor and germ-free in a like tower unit with a one hand flip handle and uses plastic grocery or trash bags. Another prior art, by Safety 1^{st}, claims a Diaper Disposal System and is similar in nature, yet, has an easy pull handle so there is no twisting and turning, with a double barrier clamp system and requires refill cartridges. Here, the prior art does claim a way and means for discarding and/or disposing of a soiled diaper after-use. However, the problem with the prior arts' claims is that, their products require expensive equipment and expensive bag cartridge refills for unnecessary and additional costs to the consumer.

Also, these types of disposal systems can be problematic in that they are not easy to change out the bag cartridges and are many times too time consuming. Another problem with the prior art is one of inconvenience wherein, to empty the stored contents of the unit when filled-up or otherwise full, a person has to lift the unit up and take out the stored contents from the bottom of the unit, which is awkward and heavy. Furthermore, the Plastic tube-bag incorporated into the operation of the prior art must be cut and the unit provides no such cutting device and this also being difficult and inconvenient. Also, products of the prior art must be located in the room or area where the used diaper products are to be discarded. Even more important, another problem with the prior art is the size of the bag(s) and/or tube-bag they use in their disposal system which is a safety hazard to a small child who could easily entangle themselves, suffocate and/or could choke, if the bags they use were to become within the reach of an unsupervised child by way of an adult's carelessness or too busy to keep within the confines of such waste disposal systems' base.

Furthermore, all of these disposal systems require an independent bag type system that is not joined directly to the equipment at all times, such as the example, where the cartridge is purchased at a store, grocer bagged, brought home, taken out of the grocer bag, and placed on the counter, floor, a table, shelf, in a cabinet or the like. More detrimental than the bag cartridge is the above related art wherein, the disposal system utilizes grocer bags that are inconvenient to store away and in most cases are stored under a kitchen sink cabinet or other area(s) that may be accessible to children within the home. Thereby, the above-mentioned dangers and hazards to an unsupervised child are always present.

Accordingly, it would be desirable to provide a disposable absorbent article(s) such as diaper, with a built-in, self-containing disposal system, which includes a joined and or bonded compactly stored pull-out sealable storage bag within a pouch or under an over-layer of material that is protected by a discreet integrated child-resistant safety tab or tape, that is disposed discreetly and directly to the center of the back waist region on an outer surface and/or backsheet of the disposable absorbent article. Further, it being effective for storing, sealing and containment of odor and germs of the folded article that is soiled with urine, feces and or other body fluids, thereby, concealing the odor and germs of the used disposable article in a convenient, always available, closed odor and germ-resistant sealable bag for proper and safe discard and or disposal that is more environmental compatible.

### SUMMARY OF INVENTION

In order to better contain and manage viscous fluid bodily wastes and the odor and germs after the disposable absorbent article's use by the wearer, the present invention provides a discreetly placed and integrated, joined and/or bonded, and compactly stored pull-out liquid impermeable and sealable bag within a pouch or under an over-layer of material that is protected by a discreet child-resistant safety tab or tape, that is disposed, joined and or bonded discreetly and directly to the upper back waist region on a surface and/or layer of the diaper, for storing, sealing and containment of odor and germs of the used folded diaper that is soiled with urine, feces and or other body fluids, thereby, concealing the odor and germs of the used diaper in a closed odor and germ-resistant sealable bag for proper and safe discard and or disposal of the diaper. Several benefits are realized by the present invention in that the article containing viscous fluid bodily wastes is contained, odor resistant for keeping room or areas fresh smelling, the storage of the article is germ resistant keeping room or area germ free, convenience of a compactly stored disposable throw away storage bag always available regardless of where the change of article occurs, it's truly portable, easy to use, anytime, anywhere, indoors and/or outdoors, alleviates unnecessary hazardous and dangerous independent and expensive diaper disposal equipment and refill cartridges, can be used traveling in an auto, airplane, train, bus, boat, etc., useful at a restaurant, stores, daycare, school, church, parks, beach, and visits to family and friends' homes, thereby, rendering the present invention environmentally compatible, inexpensive and extremely convenient. The manufacture of the invention would include mostly materials already used in the manufacture of the disposable articles thereby incorporating the invention efficiently and economically.

As noted above, the present invention is applicable to many types of disposable absorbent articles such as diapers, training pants, incontinence briefs, incontinence undergarments or pads, absorbent inserts, diaper holders and liners, feminine hygiene garments, wipes, pads and the like. Thus, the following examples of preferred embodiments of the present invention should not be construed to limit the scope of the invention.

### BRIEF DESCRIPTION OF THE DRAWINGS

FIG. 1 is a plan view of an absorbent article in its flat-out state, with embodiment of the present invention.

FIG. 1A is a plan view of the exterior upper back waist region of an absorbent article in accordance with the present invention, which includes a pliable safety tab and safety strip, the backside outer exterior surface of the diaper facing the viewer.

FIG. 1B is a plan view of the exterior upper back waist region of an absorbent article in accordance with the present invention, having a layer partially removed to reveal the underlying structure, the backside outer exterior surface of the diaper facing the viewer.

FIG. 1C is a plan view of the exterior upper back waist region of an absorbent article in accordance with the present invention, having a layer partially removed to reveal the underlying structure and pliable storage bag, the backside outer exterior surface of the diaper facing the viewer.

FIG. 2 is a plan view of the exterior upper back waist region of an absorbent article in accordance with the present invention, with an alternative embodiment of the present invention, the backside outer exterior surface of the diaper facing the viewer.

FIG. 2A is a plan view of the exterior upper back waist region of an absorbent article in accordance with FIG. 2 of the present invention, having a layer partially removed to reveal the underlying structure, the backside outer exterior surface of the diaper facing the viewer.

FIG. 2B is a plan view of the exterior upper back waist region of an absorbent article in accordance with FIG. 2 of the present invention, having a layer partially removed to reveal the underlying structure and pliable storage bag, the backside outer exterior surface of the diaper facing the viewer.

### DEFINITIONS

Within the context of this specification, each term or phrase below will include the following meaning or meanings:
(a) “Absorbent Article” refers to devices that absorb and contain body exudates, and more specifically, refers to devices that are placed against or in proximity to the body of the wearer to absorb and contain the various exudates discharged from the body.
(b) “Disposable” refers to absorbent articles that generally are not intended to be laundered or otherwise restored or reused as an absorbent article (i.e., they are intended to be discarded after a single use and, preferably, to be recycled, composted or otherwise disposed of in an environmentally compatible manner).
(c) “Disposed” refers to an element(s) of the diaper is formed, joined and positioned in a particular place or position as a unitary structure with other elements of the diaper or as a separate element joined to another element of the diaper.
(d) “Compactly Stored” refers to and encompasses configurations whereby an element is relatively small, light, economical and is closely and firmly packed, compressed or put together, taking little space and/or arranged neatly in a small space.
(e) “Bonded” refers to the joining, adhering, connecting, attaching, or the like, of two elements. Two elements will be considered to be bonded together when they are bonded directly to one another or indirectly to one another, such as when each is directly bonded to intermediate elements.
(f) “Integrated” refers to various portions of a single unitary element rather than separate structures bonded to or placed with or placed near one another.
(g) “Joined” refers to and encompasses configurations whereby an element is directly secured to another element by affixing the element directly to the other element, and configuration whereby an element is indirectly secured to another element by affixing the element to intermediate member(s) that in turn are affixed to the other element.
(h) “Seal” refers to and encompasses an element that seals, closes, shuts, or fastens tightly or securely, as against the passage of air, gases or water.
(i) “Unitary Article” refers to articles that are formed of separate parts united together to form a coordinated entity so that they do not require separate manipulative parts like a separate liner or holder.
(j) “Diaper” refers to an absorbent article generally worn by infants and incontinent persons about the lower torso, and/or disposable absorbent article worn by pets.
(k) “Layer” when used in the singular can have the dual meaning of a single element or plurality of elements.
(l) “Surface” includes any layer, film, woven, unwoven, laminate, composite, or the like, whether pervious or impervious to air, gas, and/or liquids.
(m) “Liquid Impermeable” when used in describing a layer or outer-layer laminate means that a liquid, such as urine, will not pass through the layer or laminate, under ordinary use conditions, in a direction generally perpendicular to the plane of the layer or laminate at the point of contact. Liquid or urine may spread or be transported parallel to the plane of the liquid impermeable layer or laminate.
(n) “Liquid Permeable” refers to a material present in one or more layers, such as a film, non-woven fabric, or open-celled foam, which is porous, and which is water permeable due to the flow of water and other aqueous liquids, such as urine, through the pores.
(o) “Woven” and “Woven Web” refer to materials and webs of material that are formed with the aid of textile weaving or knitting process.
(p) “Meltblown” refers to fibers formed by extruding a molten thermoplastic material through a plurality of fine, usually circular, die capillaries as molten threads or filaments into converging high velocity heated gas (eg., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al.
(q) “Pliable” refers to materials which are compliant and which will readily conform to the general shape and contours of the wearer's body.
(r) “Polymers” include, but are not limited to, homopolymers, copolymers, such as for example, block, graft, random and alternating copolymers, terpolymers, etc. and blends and modifications thereof Furthermore, unless otherwise specifically limited, the term “polymer” shall include all possible geometrical configurations of the material. These configurations include, but are not limited to isotactic, syndiotactic and atactic symmetries.

### DETAILED DESCRIPTION OF THE PREFERRED EMBODIMENTS

In accordance with the present invention, it has been discovered that a disposable and pliable storage bag integrated within or under a pliable storing pouch thereof can be strategically positioned in a disposable absorbent article such as, a child's disposable diaper or other disposable undergarment such that the wearer cannot feel nor detect its presence, and whereby, the wearer's use of the disposable article has been soiled or contains bodily exudates, the contaminated absorbent article is conveniently placed into an attached portable storage bag for immediate disposal in a more environmental compatible manner, thereby alleviating unnecessary messes, unpleasant odor and germs. Although discussed primarily in the context of a child's disposable diaper, it should be understood that the present invention and its functions are also applicable to children's disposable training pants, pull-ups, swim pants, and adult personal care products such as in absorbent incontinence undergarments and the like.

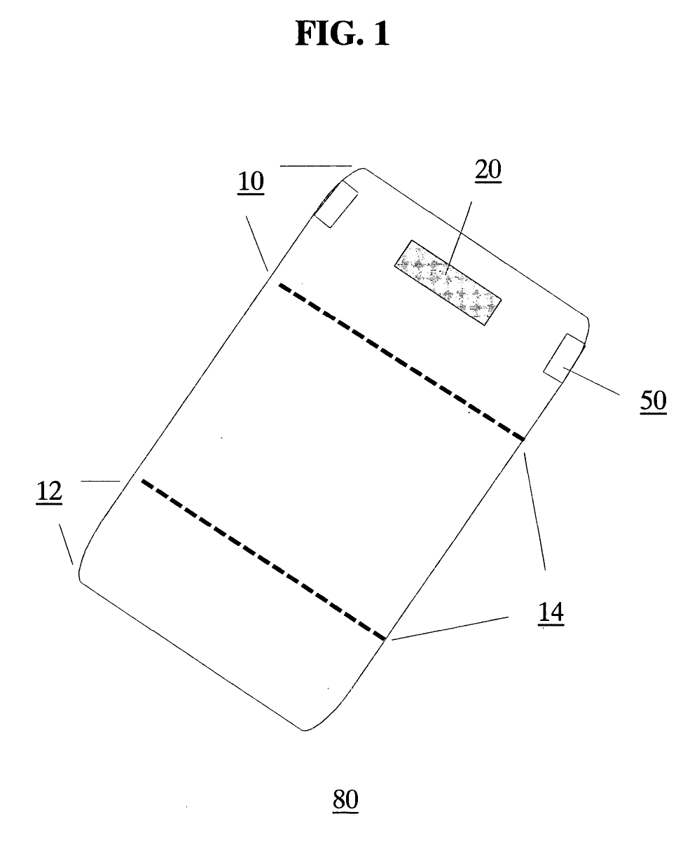

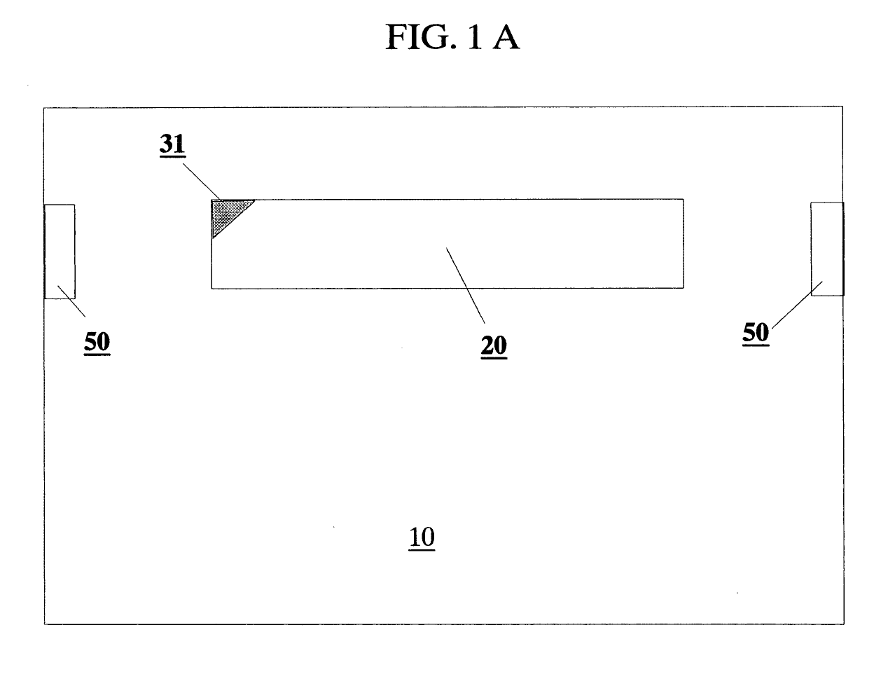

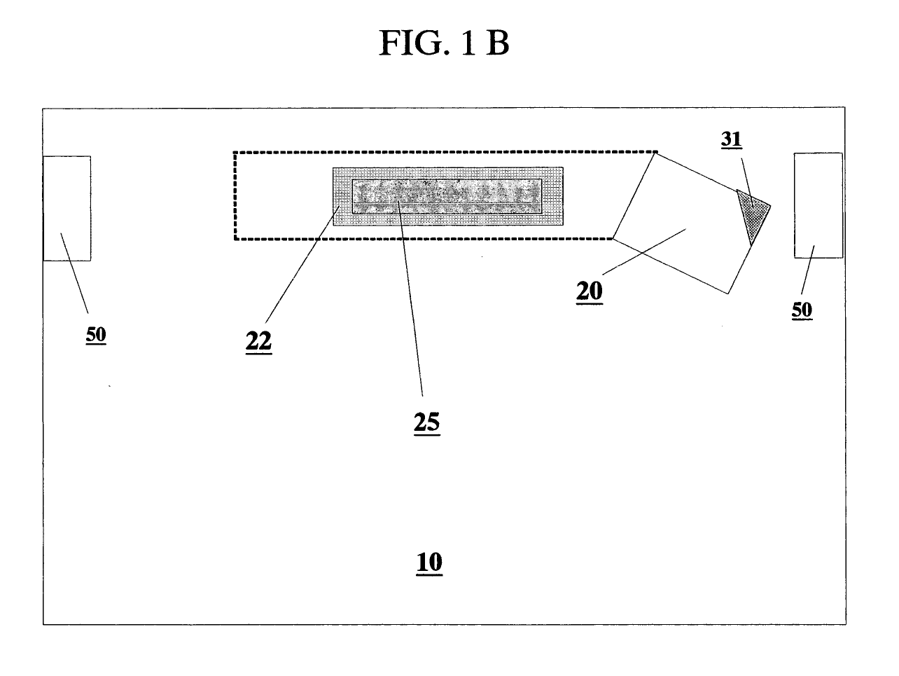

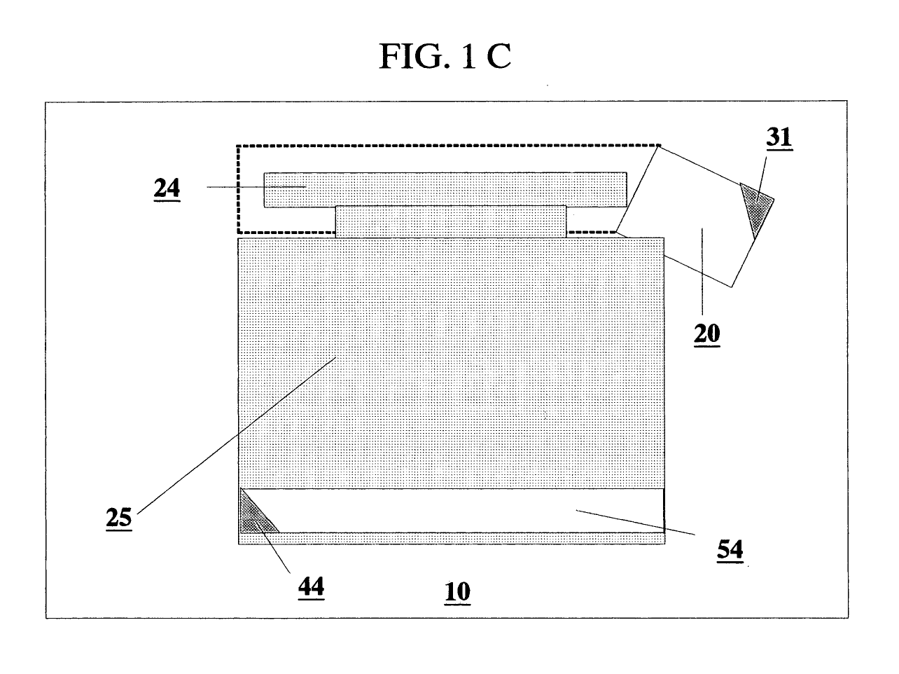

Referring to FIG. 1, is a plan view of a preferred embodiment of a disposable absorbent diaper 80 in its flat-out state in accordance with the present invention, the backside outer exterior surface facing the viewer. Further shown are a back waist region 10, a front waist region 12, and a crotch region 14. The back waist region 10 also including two provided diaper fastening tape tabs 50 illustrating the area of proximity to be the upper-side of back waist region 10 where the pliable safety strip 20 is disposed on center of an exterior Liquid impermeable surface or layer in the horizontal direction. The safety strip 20 may be manufactured in a wide variety of sizes and shapes (e.g., square, round, rectangular, hourglass, “T”-shaped, asymmetric, etc.).

The front waist region 12 is shown for illustration purposes only, in that when the disposable diaper 80 has been contaminated with urine or other bodily exudates, the front waist region 12 of the disposable diaper 80 is generally used to commence the wiping and/or partial cleaning of the wearer's body wherein, a parent or caregiver wipes the penis, vagina and anus areas of the wearer that might be covered with urine or feces contained in the outermost interior layer of the crotch region 14 of the disposable diaper 80. Furthermore, it will be recognized by one skilled in the art that the parent or caregiver, generally, then proceeds to use wipes, wet-wipes or other disposable material to finish cleaning the wearers body, placing the used wipe(s) into the outermost interior layer of the crotch region 14 of the soiled disposable diaper 80, and thereby continues to roll-up and/or fold-up the front waist region 10 into the crotch region 14 in an inwardly direction towards the interior of the back waist region 10 into a ball-like object and re-fastening the two tape tabs 50 to the outer-most exterior back waist region 10 for securing the disposable diaper 80 to then be discarded.

FIG. 1A, is a plan view of the preferred embodiment of the upper exterior side of the back waist region 10 of the disposable absorbent diaper 80 in accordance with the present invention as earlier referenced in FIG. 1, further showing a pliable safety strip 20 disposed in the center of the article in the horizontal direction of the upper portion of the exterior back waist region 10, demonstrating the pliable safety strip 20 in a closed, sealed, and or locked position. Further, the incorporation of a pliable safety tab 31 is a child-resistant locking and or gripping apparatus with a functional design disallowing and or deterring a small child from opening the peel and/or pull away safety strip 20. In furtherance of this subject matter, the selection for placement of the present invention in the back waist region 10 also aids in non-tampering of the invention in that, generally and most always the back waist region 10 is out of the view of the wearer such as, an infant, toddler, or other small child.

The pliable safety strip 20 having a primary distal end that is secure and non-removable and a second distal end that is removable. The pliable safety strip 20 and the pliable safety tab 31 to be a unitary article made from materials such as an aggressive bonding adhesive, glue, hook and loop, polymers, Meltblown materials, etc, and/or other material already being used by manufacturers within the disposable diaper industry, such as, the diaper tape tabs 50 which are utilized for gripping, attaching, bonding etc. A more descriptive and detailed discussion of material tolerances and method of manufacturing of such, are described in U.S. Pat. No. 6,267,836 entitled “Method of manufacturing a tape tab having a rounded user's end” issued to Fenske et al., on Jul. 31, 2001, assigned to The Procter & Gamble Company (Cincinnati, Ohio), which is incorporated herein by reference. The present invention in neat form, encapsulated form, or a combination thereof, may be introduced into the liner, layer or surface of the disposable absorbent article 80 and may be embossed and/or matte finished to provide a more cloth like appearance.

FIG. 1B, is a plan view of the preferred embodiment of the upper back waist region 10 of an absorbent article in accordance with the present invention, having a portion of the safety strip 20 pulled away as to demonstrate the function of the second horizontal distal end of the safety strip 20 being movable and the primary horizontal distal end of the safety strip 20 being secure and non-removable. Furthermore, it reveals the underlying structure of the present invention, thereby, disclosing the concealed pliable pouch 22 compartment that is joined and/or integrated into the disposable absorbent diaper 80 and inclusively embodies the compactly stored pliable and liquid impermeable storage bag 25 that is integrated and/or bonded to an interior and/or exterior liquid impermeable layer of the disposable absorbent diaper.

As illustrated herein, the pliable safety strip 20 and the pliable safety tab 31 are constructively designed in a manner to which they are a unitary article that does not completely pull-away, tear-away and/or peel-away from the embodiment surface layer of the disposable diaper 80. This is desirable in that there are no small parts or pieces that become independent from the present invention for a infant, toddler or other child to place in there mouth and thereby become a choking hazard. In furtherance of this subject matter, the pliable pouch 22 compartment is non removable from its anchoring surface layer, and has no small parts or pieces independent from the disposable diaper 80.

The manufacture of the materials to be used in the present invention, already are widely used throughout the disposable diaper industry and industries of other disposable absorbent articles, as earlier disclosed in U.S. Pat. No. 6,267,836 entitled “Method of manufacturing a tape tab having a rounded user's end” issued to Fenske et al., on Jul. 31, 2001, assignee The Procter & Gamble Company (Cincinnati, Ohio). Another descriptive and detailed discussion in the manufacture of these materials used for manufacturing disposable absorbent articles, such as diapers and other adult incontinence undergarments, can be found in U.S. Pat. No. 3,848,594 entitled “Tape Fastening System for Disposable Diaper” issued to Buell on Nov. 19, 1974; the earlier disclosed U.S. Pat. No. 3,860,003 issued to Kenneth Barclay Buell on Jan. 14, 1975; and U.S. Pat. No. 4,846,815 entitled “Disposable Diaper Having An Improved Fastening Device” issued to Scripps on Jul. 11, 1989; U.S. Pat. No. 4,894,060 entitled “Disposable Diaper With Improved Hook Fastener Portion” issued to Nestegard on Jan. 16, 1990; U.S. Pat. No. 4,946,527 entitled “Pressure-Sensitive Adhesive Fastener And method of Making Same” issued to Battrell on Aug. 7, 1990; U.S. Pat. No. 5,151,092 issued to Buell on Sep. 9, 1992. Each of these patents is incorporated herein by reference.

In further light of the present invention being disposed on the upper back waist region 10 as referenced in FIG. 1A, it should be obvious to one skilled in the art that generally, most all disposable diapers, once contaminated with bodily exudates by the wearer, are rolled up and/or folded up by commencing with the front waist region 12 being turned in a direction inwardly toward the interior crotch region 14, and proceeding toward the interior back waist region 10, into a ball-like object and re-fastening the two tape tabs 50 to the outer-most exterior back waist region 10 for securing the disposable diaper 80 to then be discarded. Herein, respectively, an exterior layer disposed in the upper back waist region 10 is the preferred location for accessing the present invention. The present invention in neat form, encapsulated form, or a combination thereof, may be introduced into the liner, layer or surface of the disposable absorbent article 80 and may be embossed and/or matte finished to provide a more cloth like appearance.

FIG. 1C, is a plan view of a preferred embodiment of the upper back waist region 10 of an absorbent article in accordance with the present invention, having a portion of the safety strip 20 incorporating a safety tab 31, pulled away as to demonstrate the function of the second distal end of the safety strip 20 being movable and the primary distal end of the safety strip 20 being secure and non-removable. Furthermore, it reveals the underlying structure of the present invention, thereby, exposing the compactly stored pliable and liquid impermeable storage bag 25 in a flat-out position showing that the bag 25 has been pulled-out to be utilized, and is in an inverted and/or upside down direction from the upper side of the back waist region 10.

In this position, the inverted pliable storage bag 25 is vulnerable to a person placing one hand inside the pliable storage bag 25, likened to a person putting there hand into a glove, and grabbing the folded and/or rolled up contaminated disposable diaper 80 thereby, encapsulating the disposable diaper 80 by use of the person's other hand, by pulling the effective open area of the inverted storage bag up and over and around the article so as to re-invert the storage bag 25, now fully encapsulating the folded or rolled up disposable article 80, thereby reversing the original exterior surface of the storage bag 25 to an interior position and the original interior surface of the storage bag 25 now the exterior surface of the storage bag 25.

Also illustrated is the pliable safety retainer strip 24 that is integrated within a portion of the pliable storage bag 25 and bonded directly to a surface layer of the disposable diaper 80 which serves as a retaining and/or retention source thereby, the pliable storage bag 25 permanently anchored and attached to the disposable diaper 80. The safety retainer strip 24 being disposed at the bottom distal end of the inverted pliable storage bag 25 which being an effective closed area of the pliable storage bag. The illustration further demonstrating a seal tab 44 and a seal strip 54 as a unitary article integrated within a portion of the inverted pliable storage bag 25 and is further disposed at the effective open area of the inverted pliable storage bag 25. The seal strip 54 and the seal tab 44 when in a closed position renders the liquid impermeable storage bag to be air, gas and watertight. Further, the pliable storage bag 25 to be made of a material(s), film, layer(s) that are liquid impermeable, odor resistant and germ resistant. The pliable storage bag can further be treated with a chemical source scent for other odor freshness, and/or a chemical formulation of ingredients as an integral component of an anti-microbial, anti-viral, anti-bacterial or anti-fungal source ingredient.

Also, the embodied seal strip 54 can be devised or constructed in a manner, such as, a seal track of a zip-lock storage bag or sandwich bag with a seal apparatus that operates horizontally across the effective open area of the storage bag 25, and/or an adhesive layered strip disposed horizontally across the effective open area with a protective peel strip. Different layers of particles may be directly and/or indirectly adjacent each other or may be separated by one or more materials, such as netting, scrim, non-woven, woven webs, film, foam, adhesive, and the like. Again, the manufacturing of the present invention can easily be incorporated into the manufacture of existing and/or current disposable absorbent articles efficiently and economically, by means of materials, process(es), and other source of current applications already being utilized for the manufacture of disposable absorbent articles.

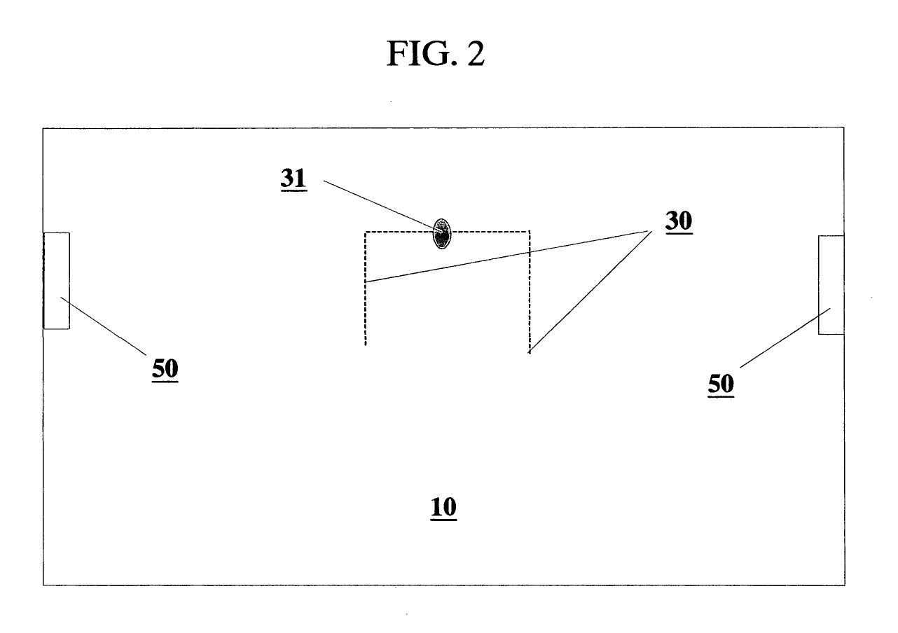

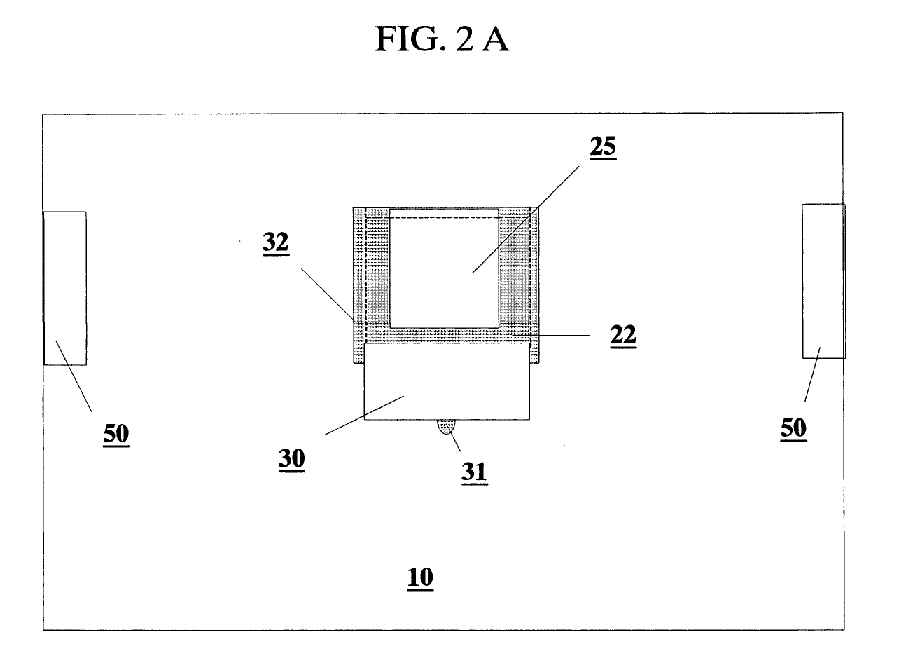

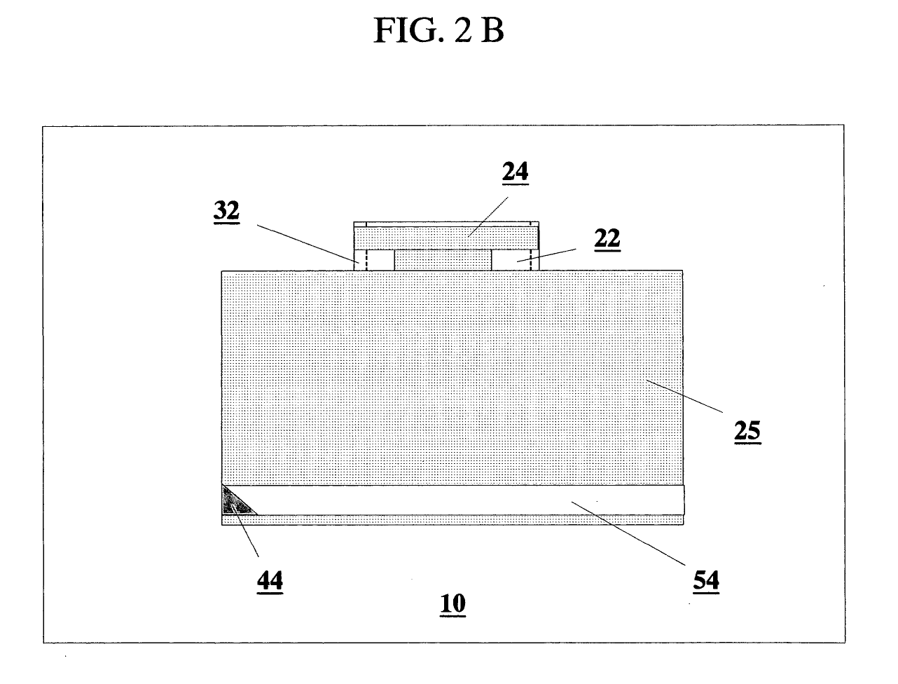

FIG. 2, is an alternative embodiment of the present invention within a plan view of the exterior upper back waist region 10 of an absorbent article in accordance with the present invention, again, the backside outer exterior surface of the diaper facing the viewer. Also, the two diaper tape tabs 50 are illustrated herein for referencing points only. In this alternative embodiment of the present invention, the earlier referenced safety strip 20 of FIG. 1A, is replaced with a shell cover 30 utilized to encapsulate the pliable storage bag 25 and wherein, the encapsulation material shell thickness may vary depending upon the element(s) of material utilized, and may be a monolayer or thicker laminate layer, or may be a composite layer of the same of disposable absorbent article 80 and wherein, the shell cover 30 is a pre-packaged pouch compartment 22 having a back sheet 32 that is bonded to the outer most exterior surface layer of the disposable article 80 and thereby, containing the pliable storage bag 25 between the shell cover 30 and the back sheet 32 which is joined to the outer most exterior layer of the disposable absorbent article 80.

Again, the present invention in an alternative embodiment should be located or be of a size such that the wearer cannot feel its elements. The operation of the present invention in the alternative embodiment functions much the same way as the preferred embodiment of the present invention, with the exception to the pre-manufacture of the pouch compartment 22 shown in FIG. 2A, comprising the shell cover 30 and the safety tab 31 and the back sheet 32 shown in FIG. 2A, and the compactly stored pliable storage bag 25, all inclusively are later bonded, joined and/or integrated to the absorbent article 80. Although in the alternative embodiment of the present invention, it is illustrated where the safety tab 31 is deposed in a vertical or longitudinal direction, it may also be disposed and the function thereof being a horizontal direction and the operation and function compatible therein. It should be recognized, however, that the pliable storage bag 25 and the pliable pouch 22 shown in FIG. 2A, and the safety tab 31 and the shell cover 30 thereof, may be located on or within the outer layer, or any other layer or layers comprising the disposable article 80 and may be embossed and/or matte finished to provide a more cloth like appearance.

FIG. 2A, is again referencing an alternative embodiment of the present invention within a plan view of the exterior upper back waist region 10 of an absorbent article 80 in accordance with the present invention, illustrating the shell cover 30 having a portion pulled away as to demonstrate the function of the safety tab 31 and the second longitudinal distal end of the shell cover 30 being movable and the primary longitudinal distal end of the shell cover 30 being secure and non-removable. Also, the two diaper tape tabs 50 are illustrated herein for referencing points only.

Furthermore, it reveals the underlying structure of an alternative embodiment of the present invention and discloses a back sheet 32 that is joined and/or integrated into and/or onto a surface or layer of the disposable absorbent diaper 80 and inclusively embodies the compactly stored pliable and liquid impermeable storage bag 25 that is integrated and/or bonded between a top layer of the back sheet 32 and beneath a back layer and/or surface of the pliable shell cover 30 bonded or joined to a top layer or surface of back sheet 32 or a surface layer of absorbent article 80. As illustrated herein, the pliable shell cover 30 and the pliable safety tab 31 are constructively designed in a manner to which they are a unitary article that does not completely pull-away, tear-away and/or peel-away from the embodiment surface layer of the disposable diaper 80. The alternative embodiment in accordance with the present invention in neat form, encapsulated form, or a combination thereof, may be introduced into the liner, layer or surface of the disposable absorbent article 80.

FIG. 2B, further referencing an alternative embodiment of the present invention within a plan view of the exterior upper back waist region 10 of an absorbent article 80 in accordance with the present invention, reveals the underlying structure of the present invention, thereby, exposing the pouch compartment 22 encompassing the compactly stored pliable and liquid impermeable storage bag 25 in a flat-out position and/or showing the storage bag 25 has been pulled-out to be utilized, and is in an inverted and/or upside down direction from the upper side of the back waist region 10.

In this position, the inverted pliable storage bag 25 is vulnerable to a person placing one hand inside the pliable storage bag 25, likened to a person putting there hand into a glove, and grabbing the folded and/or rolled up contaminated disposable diaper 80 thereby, encapsulating the disposable diaper 80 by use of the person's other hand, by pulling the effective open area of the inverted storage bag up and over and around the article so as to re-invert the storage bag 25, now fully encapsulating the folded or rolled up disposable article 80, thereby reversing the original exterior surface of the storage bag 25 to an interior position and the original interior surface of the storage bag 25 now the exterior surface of the storage bag 25.

The safety retainer strip 24 being disposed at the bottom distal end of the inverted pliable storage bag 25 which being an effective closed area of the pliable storage bag. The illustration further demonstrating a seal tab 44 and a seal strip 54 as a unitary article integrated within a portion of the inverted pliable storage bag 25 and is further disposed at the effective open area of the inverted pliable storage bag 25. The seal strip 54 and the seal tab 44 when in a closed position renders the liquid impermeable storage bag to be air, gas and watertight. Further, the pliable storage bag 25 to be made of a material(s), film, layer(s) that are liquid impermeable, odor resistant and germ resistant. The pliable storage bag can further be treated with a chemical source scent for other odor freshness, and/or a chemical formulation of ingredients as an integral component of an anti-microbial, anti-viral, anti-bacterial or anti-fungal source ingredient.

Further illustrated is the pliable safety retainer strip 24 that is integrated within a portion of the pliable storage bag 25 that is integrated and/or bonded between a top layer of the back sheet 32 and/or a top surface layer of the pouch compartment 22 and/or a top surface layer of disposable absorbent article 80, which serves as a retaining and/or retention source thereby, the pliable storage bag 25 permanently anchored and attached to the disposable diaper 80. Also, the embodied seal strip 54 can be devised or constructed in a manner, such as, a seal track of a zip-lock storage bag or sandwich bag with a seal apparatus that operates horizontally across the effective open area of the storage bag 25, and/or an adhesive layered strip disposed horizontally across the effective open area with a protective peel strip. Different layers of particles may be directly and/or indirectly adjacent each other or may be separated by one or more materials, such as netting, scrim, non-woven, woven webs, film, foam, adhesive, and the like. Again, the manufacturing of the present invention can easily be incorporated into the manufacture of existing and/or current disposable absorbent articles efficiently and economically, by means of materials, process(es), and other source of current applications already being utilized for the manufacture of disposable absorbent articles.

While particular embodiments of the present invention have been illustrated and described, it would be obvious to those skilled in the art that various other changes and modifications can be made without departing from the spirit and scope of the invention. It is therefore intended to cover in the apprehended claims all such changes and modifications that are within the scope of this invention.

1. A disposable absorbent article comprising:
a back-waist region;
a front-waist region; and
a crotch region extending between the front waist region and the back waist region

2. The disposable absorbent article of claim 1 comprising:
a liquid impermeable exterior surface layer;
a liquid impermeable interior surface layer;
a liquid permeable interior surface layer; and
a liquid absorbent core disposed in the crotch region between the exterior surface layer and the interior surface layer;

3. The disposable absorbent article of claim 1 comprising a joined and/or integrated pliable pouch and/or an inner compartment for storage;

4. The disposable absorbent article of claim 3 wherein the pliable pouch and/or compartment is disposed in the center of disposable absorbent article of claim 1, in the horizontal direction of the upper portion of back waist region;

5. The disposable absorbent article of claim 4 wherein the pliable pouch and/or compartment is disposed on and/or in a liquid impermeable exterior surface layer;

6. The disposable absorbent article of claim 1 comprising a joined and/or integrated pliable and removable safety strip, patch, tape and or like source;
a distal end of safety strip permanently applied and non-removable;
a second distal end of safety strip applied temporarily for removal;

7. The disposable absorbent article of claim 6 comprising a joined and/or integrated pliable safety tab and/or apparatus;

8. The disposable absorbent article of claim 3 wherein the pliable pouch and/or compartment having perforated markings on a liquid impermeable exterior surface layer, in a horizontal direction and/or a longitudinal direction for opening;

9. The disposable absorbent article of claim 3 wherein the pliable pouch and/or compartment is made of a material of the same as said disposable absorbent article of claim 1; and
made of such other compatible material, as tape or other type of bond adhesive patch or shell cover;

10. The disposable absorbent article of claim 1 comprising a liquid impermeable, odor resistant and germ resistant pliable storage bag;

11. The disposable absorbent article of claim 10 wherein the liquid impermeable and pliable storage bag is integrated and/or bonded to either a back sheet, layer or surface;

12. The disposable absorbent article of claim 10 wherein the liquid impermeable and pliable storage bag is compactly stored within the disposable article of claim 3;

13. The disposable absorbent article of claim 10 wherein the liquid impermeable and pliable storage bag comprising:
an effective open area; and
an effective closed area;

14. The disposable absorbent article of claim 10 wherein the liquid impermeable and pliable storage bag further comprising:
a seal tab; and
a seal strip, seal line, or seal track source;

15. The disposable absorbent article of claim 10 wherein the liquid impermeable and pliable storage bag being disposed in the center of an interior surface layer of disposable absorbent article of claim 1 in the horizontal direction of the upper portion of back waist region;

16. The disposable absorbent article of claim 15 wherein the liquid impermeable and pliable storage bag being disposed under and/or within an exterior surface layer of claim 3;

17. The disposable absorbent article of claim 10 wherein the liquid impermeable and pliable storage bag is disposed in an inverted position, comprising:
a flipped or turned-out and/or inside-out original position;
a reversible position;
an upside-down position;

18. The disposable absorbent article of claim 10 wherein the liquid impermeable and pliable bag comprising an integrated safety retainer strip; and/or
other pliable retaining source;

19. The disposable absorbent article of claim 10 wherein the liquid impermeable and pliable bag comprising:
a chemical source scent treatment, and/or
a chemical formulation as an integral component of anti-microbial, anti-viral, anti-bacterial and/or anti-fungal source.
